(19) United States
(12) Patent Application Publication
(10) Pub. No.: US 2026/0266174 A1
(43) Pub. Date: Sep. 10, 2026

(54) DEVICE AND METHOD FOR EVALUATING SEEPAGE CORROSION AND MECHANICAL PROPERTIES OF CEMENT SHEATH FOR CEMENTING

(71) Applicant: Southwest Petroleum University, CHENGDU CITY (CN)

(72) Inventors: Kaiqiang LIU, CHENGDU CITY (CN); Xingguo ZHANG, CHENGDU CITY (CN); Yuanpeng WU, CHENGDU CITY (CN); Wenli TANG, CHENGDU CITY (CN); Ji ZHOU, CHENGDU CITY (CN)

(21) Appl. No.: 19/363,942

(22) Filed: Oct. 21, 2025

(30) Foreign Application Priority Data
Mar. 6, 2025 (CN) 202510262801.3

(51) Int. Cl. E21B 47/005; G01N 33/38

(52) U.S. Cl. CPC E21B 47/005; G01N 33/383

(57) ABSTRACT

The present invention discloses a device and method for evaluating seepage corrosion and mechanical properties of a cement sheath for cementing. The device includes an acidic corrosive gas injection system, a formation water injection system, a high-pressure oil injection system, a control system and a corrosion and test system, wherein the corrosion and test system includes a main body, an upper cover and a base; the main body sequentially includes a metal cylinder, a simulated formation, a simulated casing and a center rod from outside to inside; a cement sheath curing-corrosion space is formed between the simulated formation and the simulated casing; a temperature control device is arranged in an annulus formed between the simulated casing and the center rod; and the center rod is provided with a plurality of displacement meters at different positions and angles to measure a deformation displacements of a simulated casing-cement sheath assembly.

### FIELD OF THE INVENTION

The present invention belongs to the technical field of corrosion evaluation of the cementing materials in hydrocarbon wells, and in particular, to a device and method that can achieve high-temperature and high-pressure curing of a cement sheath, seepage corrosion of the cement sheath and a cement sheath-formation interface and mechanical integrity evaluation.

### BACKGROUND OF THE INVENTION

In the cementing engineering of hydrocarbon wells, a cement sheath, as a key part of a shaft, plays a role in supporting a casing and isolating between formation layers, thereby ensuring the safe and efficient exploitation of oil and gas resources. However, compared with a metal casing of a shaft and a formation, a cement sheath has the weakest mechanical properties and is thus extremely prone to becoming the weak link causing the failure of shaft sealing integrity. Meanwhile, the cement sheath also faces high temperature and high pressure at the bottom of a well and a corrosion environment of formation fluids. Especially in oil and gas wells with high H₂S/CO₂ content and CO₂ sequestration wells, a neutralization reaction occurs between acidic H₂S and CO₂ and an alkaline cement sheath, which will change a chemical-microstructure of cement sheath, destroy the cementing quality between a cement sheath body and a cement sheath-formation interface, and cause the failure of an interlayer isolation effect of the cement sheath. Therefore, it is urgent to accurately grasp the mechanical properties and permeability evolution law of cement sheath in a high-temperature and high-pressure-corrosive medium, so as to provide means and support for the evaluation of the corrosion resistance of cement sheath and the design of corrosion-resistant cement slurry in oil and gas wells with high-acid-content media and CO₂ sequestration wells.

In recent years, scholars have focused on the corrosive effect of acidic media on cement paste by establishing corrosion methods. In “Dynamic Corrosion Device for Migration of High-temperature and High-pressure Corrosion Medium” (CN109520878A), a corrosion kettle is turned over by rotating the device to simulate a flow replacement of a corrosive medium on the surface of cement paste, but this device cannot reflect a comprehensive process of corrosion between the corrosive medium and the interior of the cement paste that needs to undergo seepage followed by reaction. In “Device and Method for High-temperature and High-pressure Seepage Corrosion Simulation Experiment” (CN118362491A), by allowing a corrosive medium to seep longitudinally on cement paste and through the corrosion reaction, the process of downhole seepage corrosion of cement paste under high temperature and high pressure can be simulated, and cement paste samples can be cured under seepage corrosion conditions. In “Seepage Corrosion Device and Seepage Corrosion Method for Two Interfaces of Cement Stone for Cementing” (CN118029947A), the corrosion processes of gas and liquid phases are simulated at the same time, and the simulation of high pressure at the bottom of a well and corrosion of cement paste in various corrosion modes can be achieved by applying a confining pressure to an artificial well wall and adjusting a gas-liquid corrosive medium. Further, considering that cement sheath at the bottom of a well which is formed as a ring may affect corrosion evaluation results, in “Test Device and Method for Corrosion Integrity of Cement Sheath/Plug in Carbon Dioxide Under High Temperature and High Pressure” (CN118190766A), the focus is on simulating corrosion curing of a cement sheath/cement plug for well shut-in by injecting CO₂ into a formation after shut-in of a CCUS well, liquid is injected at the upper end of the cement plug to observe bubbles, and the sealing performances of the cement sheath and the cement plug are monitored. In “Corrosion Test Device and Method for Full-scale Simulation of Cement Sheath for Cementing Under Real Downhole Conditions” (CN114878448A)”, cement slurry is injected between two metal pipes to form a cement sheath, a high-pressure corrosive medium is then injected into the end surface of the cement sheath, one-way dynamic corrosion curing is carried out for full-size cement sheaths, a cement paste core is obtained by drilling on the cement sheath at the end of corrosion, and performance indicators such as compressive strength, permeability and corrosion depth are tested.

The existing corrosion device focuses on the corrosion curing of a cement sheath in a corrosive medium under high temperature and high pressure, the cement sheath is dismantled after temperature and pressure reduction, and other devices are then used to evaluate its mechanical properties. The cement sheath is a multi-component, porous and brittle material, and the processes of temperature and pressure reduction may lead to microstructure-properties changes. Moreover, the material properties of the cement sheath and the metal casing are different, and there is a significant difference in their thermal expansion coefficients, resulting in the destruction of the structure and performance of the cement sheath during the processes of temperature and pressure reduction.

With the development of technologies such as CO₂ geological sequestration and oil displacement, higher challenges have been brought to corrosion devices and evaluation technologies for cement sheaths for cementing engineering. The existing devices have single functions and cannot accurately evaluate a corrosion status of a cement sheath under downhole conditions and the influence law of corrosion on the mechanical integrity of the cement sheath.

### SUMMARY OF THE INVENTION

An object of the present invention is to provide a device for evaluating seepage corrosion and mechanical properties of a cement sheath for cementing engineering. This device can achieve three functions of curing of a cement sheath under high temperature and high pressure, corrosion of an H₂S/CO₂ acidic medium on a cement sheath body and a formation-cement sheath interface, and evaluation of mechanical integrity of the cement sheath after corrosion, and has a wide application prospect.

Another object of the present invention is to provide a method for evaluating seepage corrosion and mechanical properties of a cement sheath by using the above-mentioned device. The method is reliable in principle and easy to operate, and solves the problem that the existing corrosion device cannot fully reflect the seepage corrosion of a cement sheath body and a formation-cement sheath interface in a formation, and cannot directly evaluate the mechanical integrity of the cement sheath after corrosion under high temperature and high pressure.

To fulfill said technical objects, the present invention adopts the following technical solutions.

A device for evaluating seepage corrosion and mechanical properties of a cement sheath includes an acidic corrosive gas injection system, a formation water injection system, a high-pressure oil injection system, a control system and a corrosion and test system.

The acidic corrosive gas injection system includes a gas cylinder, a control valve, a gas booster pump and a check valve, in which the gas booster pump is connected to the control system; the formation water injection system includes a liquid storage tank, a liquid booster pump and a check valve, in which the gas booster pump is connected to the control system; and an outlet end of the acidic corrosive gas injection system and an outlet end of the formation water injection system are respectively equipped with the check valve, converged to a high-pressure pipeline, and connected to the corrosion and test system.

The high-pressure oil injection system includes an oil storage tank, a high-pressure oil pump and a check valve; the high-pressure oil pump is connected to the control system; and an outlet end of the high-pressure oil injection system is equipped with the check valve, and connected to the corrosion and test system.

The corrosion and test system includes a main body, an upper cover and a base; the main body sequentially includes a metal cylinder, a simulated formation, a simulated casing and a center rod from outside to inside; the center rod is located in the center of the base and connected to the upper cover through a nut; the metal cylinder forms an effective seal with the simulated formation after epoxy resin is solidified; a cement sheath curing-corrosion space is formed between the simulated formation and the simulated casing; a temperature control device is arranged in the annulus formed between the simulated casing and the center rod; the temperature control device is connected to the control system and used to adjust a temperature in the corrosion and test system.

An injection port and an oil inlet are formed in positions of the base corresponding to the simulated formation and the simulated casing-center rod annulus, respectively; the injection port is connected to the formation water injection system and the acidic corrosive gas injection system, respectively; and the oil inlet is connected to the high-pressure oil injection system.

A discharge port and an oil outlet are formed in positions of the upper cover corresponding to the simulated formation and the simulated casing-center rod annulus, respectively; and the discharge port and the oil outlet are connected to a back pressure valve through a high-pressure pipeline, respectively.

The simulated casing is used to simulate a casing in a cementing operation, and plays a role in supporting a well wall and preventing oil and gas leakage.

The center rod is provided with a plurality of (not less than 3) displacement meters at different positions and angles. The stress-strain of a material is calculated by measuring deformation displacements of the simulated casing-cement ring assembly under stress.

The acidic corrosive gas injection system and the formation water injection system provide an acidic corrosive gas and formation water for the corrosion and test system through the injection port.

The high-pressure oil injection system injects high-temperature resistant oil into the corrosion and test system through the oil inlet to control a pressure of the simulated casing-center rod annulus, thereby achieving pressure control during high-temperature and high-pressure curing and mechanical property test of the cement sheath.

The control system is used to control temperature and pressure conditions during curing and corrosion of the cement sheath for cementing operation, and control a pressure during mechanical property test.

The corrosion and test system is used to cure and corrode the cement sheath under high temperature and high pressure, and evaluate the mechanical properties of the cement sheath after corrosion.

The simulated formation is processed by preferably selecting outcrop rock samples according to actual formation data, or is made of artificial rock samples having parameters close to those of an actual formation.

A method for evaluating seepage corrosion and mechanical properties of a cement sheath by using the above-mentioned device includes the following steps:
(1) fixing the simulated formation in the metal cylinder by using epoxy resin, forming an effective seal after the epoxy resin is solidified, and installing the center rod, the simulated casing, and the metal cylinder containing the simulated formation on the base, respectively;
(2) injecting cement slurry into a space between the simulated formation and the simulated casing, installing the upper cover, and then tightening the upper cover by using a nut;
(3) injecting high-temperature resistant oil into the simulated casing-center rod annulus by opening the high-pressure oil injection system, injecting formation water into the simulated formation by opening the formation water injection system, and closing the back pressure valve if there is liquid flowing out of the oil outlet and the discharge port; setting a pressure of the back pressure valve, raising a temperature of the temperature control device to a required temperature through the control system, and starting to cure the cement sheath to form a simulated casing-cement sheath assembly;
(4) after the curing is completed, introducing an acidic corrosive gas H₂S/CO₂ into the corrosion and test system for corrosion testing by opening the acidic corrosive gas injection system;
(5) after the corrosion is completed, injecting a pressure into the simulated casing-center rod annulus at a certain rate through the control system and the high-pressure oil injection system to form a pressure difference between the simulated casing-cement sheath assembly and the simulated formation, recording deformation displacements generated by the simulated casing-cement sheath assembly under the pressure difference through the radial displacement meters, collecting data and drawing a stress-strain curve, and analyzing mechanical properties of the cement sheath;
(6) reducing the temperature and pressure at the end of the experiment, unscrewing the nut, opening the upper cover and taking the cement sheath out, testing the corrosion conditions (including a microstructure, a corrosion depth, etc.) of the cement sheath and the simulated formation-cement sheath interface, and analyzing a corrosion law of the cement sheath in an H₂S/CO₂ corrosion environment.

Further, the curing and the corrosion can be carried out synchronously according to the needs of experimental simulation conditions.

Compared with the prior art, the present invention has the following beneficial effects.
(1) The present invention can achieve three functions of curing of a cement sheath under high temperature and high pressure, corrosion of the H₂S/CO₂ acidic medium on the cement sheath body and the simulated formation-cement sheath interface, and evaluation of mechanical integrity of the cement sheath after corrosion at the same time. The traditional device has a single function, and is not only cumbersome to operate, but also easy to introduce human errors since different equipment needs to be used to complete the above operations. The present invention completes multiple operations in the same equipment, significantly improves the experimental efficiency, reduces the equipment footprint, and reduces the cost investment.
(2) The problem that the existing corrosion device cannot directly evaluate the mechanical integrity of a cement sheath after corrosion under high temperature and high pressure is solved. In the present invention, the deformation displacements of the cement sheath under stress can be measured directly in a high-temperature and high-pressure environment by arranging the displacement meters in the device and applying a pressure by the control system, stress-strain data can be collected, and the mechanical properties of the cement sheath can be evaluated more accurately.

### BRIEF DESCRIPTION OF THE DRAWINGS

FIG. 1 is a schematic structural diagram of a device for evaluating seepage corrosion and mechanical properties of a cement sheath for cementing.

FIG. 2 is a schematic structural diagram of a corrosion and test system.

In drawings, reference symbols represent the following components: 100-acidic corrosive gas injection system; 110-gas cylinder; 120-control valve; 130-gas booster pump; 140-check valve; 200-formation water injection system; 210-liquid storage tank; 220-liquid booster pump; 230-check valve; 300-high-pressure oil injection system; 310-oil storage tank; 320-high-pressure oil pump; 330-check valve; 400-control system; 500-corrosion and test system; 501-center rod; 502-nut; 503-back pressure valve; 504-back pressure valve; 505-metal cylinder; 506-simulated formation; 507-cement sheath curing-corrosion space; 508-simulated casing; 509-annulus; 510-temperature control device; 511-oil outlet; 512-oil inlet; 513-injection port; 514-discharge port; 515-base; 516-upper cover; and 517-radial displacement meter.

### DETAILED DESCRIPTION OF SOME EMBODIMENTS

The technical solutions of the present invention are described clearly and completely in conjunction with the embodiments and accompanying drawings.

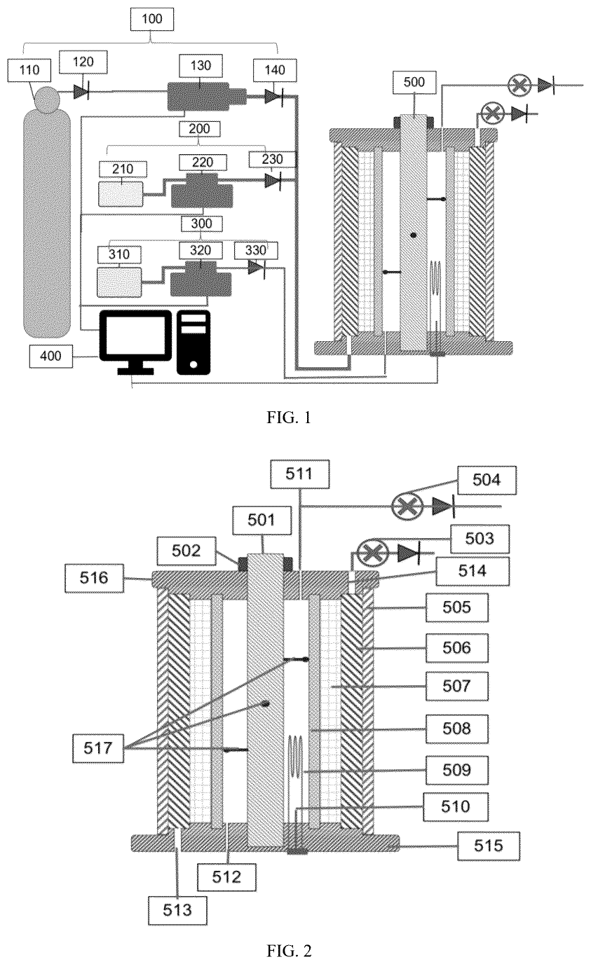

Referring to FIG. 1 and FIG. 2.

The device for evaluating seepage corrosion and mechanical properties of a cement sheath includes an acidic corrosive gas injection system 100, a formation water injection system 200, a high-pressure oil injection system 300, a control system 400 and a corrosion and test system 500.

The acidic corrosive gas injection system 100 includes a gas cylinder 110, a control valve 120, a gas booster pump 130 and a check valve 140. The formation water injection system 200 includes a liquid storage tank 210, a liquid booster pump 220 and a check valve 230. The gas booster pump and the liquid booster pump are connected to the control system 400, respectively. An outlet end of the acidic corrosive gas injection system and an outlet end of the formation water injection system are respectively equipped with the check valve, converged to a high-pressure pipeline, and connected to the corrosion and test system 500.

The high-pressure oil injection system 300 includes an oil storage tank 310, a high-pressure oil pump 320 and a check valve 330. The high-pressure oil pump is connected to the control system 400. An outlet end of the high-pressure oil injection system is equipped with the check valve, and connected to the corrosion and test system 500.

The corrosion and test system 500 includes a main body, an upper cover 516 and a base 515. The main body sequentially includes a metal cylinder 505, a simulated formation 506, a simulated casing 508 and a center rod 501 from outside to inside. The center rod is located in the center of the base and connected to the upper cover through a nut 502. The metal cylinder forms an effective seal with the simulated formation after epoxy resin is solidified. A cement sheath curing-corrosion space 507 is formed between the simulated formation and the simulated casing. An annulus 509, i.e., a simulated casing-center rod annulus, is formed between the simulated casing and the center rod. A temperature control device 510 is arranged in the annulus. The temperature control device is connected to the control system 400. An injection port 513 and an oil inlet 512 are formed in positions of the base 515 corresponding to the simulated formation and the simulated casing-center rod annulus, respectively. The injection port is connected to the formation water injection system and the acidic corrosive gas injection system, respectively. The oil inlet is connected to the high-pressure oil injection system. A discharge port 514 and an oil outlet 511 are formed in positions of the upper cover corresponding to the simulated formation and the simulated casing-center rod annulus, respectively. The discharge port and the oil outlet are connected to a back pressure valve 503, 504 through a high-pressure pipeline, respectively. The center rod 501 is provided with a plurality of radial displacement meters 517 at different positions and angles.

The device for evaluating seepage corrosion and mechanical properties of the cement sheath can achieve the curing of the cement sheath under high temperature and high pressure, seepage corrosion of a cement sheath body and a formation-cement sheath interface, and evaluation of mechanical integrity. The following specific steps are included:

Step 1: installing the metal cylinder on the base, injecting resin between the metal cylinder and the simulated formation to fill voids, preparing an H₂S/CO₂ gas cylinder, connecting all high-pressure pipelines, and selecting corresponding porous outcrop rocks or artificial cores according to a simulated formation to be tested;

Step 2: after the resin is solidified, preparing cement slurry according to GB/T 19139-2012, injecting the cement slurry into a space formed between the simulated formation and the simulated casing, and tightening the upper cover by using a nut to ensure that the sealing integrity of the device;

Step 3: injecting crude oil into the simulated casing-center rod annulus by opening the high-pressure oil injection system, injecting formation water into the simulated formation by opening the formation water injection system, and closing the back pressure valve and the check valve if the crude oil and the formation water flow out of the oil outlet and the discharge port; raising a temperature of the temperature control device to a required temperature through the control system, and curing to form a cement sheath;

Step 4: introducing an H₂S/CO₂ corrosive gas into the corrosion and test system for corrosion testing by opening the control valve and the check valve;

Step 5: after the corrosion is completed, applying a pressure to the simulated casing-center rod annulus through the control system, such that the cement sheath expends toward the simulated formation; recording deformation displacements generated by the cement sheath under stress by using the displacement meters, calculating the strain of the cement sheath, indirectly calculating the stress borne by a material in combination with known parameters such as elastic modulus of the material and according to the measured displacements and strain data, and drawing a stress-strain diagram;

Step 6: opening the upper cover and taking the cement sheath out at the end of the experiment, observing and detecting the corrosion conditions of the cement sheath to obtain the corrosion conditions of the cement sheath under H₂S/CO₂ corrosion, and analyzing a corrosion law of the cement sheath.

Obviously, the above embodiments of the present invention are only examples given to clearly illustrate the present invention, without any limitation of implementations of the present invention. For those of ordinary skill in the art, other different forms of changes or variations can be made on the basis of the above description. It is not possible to list all embodiments here, and all obvious changes or variations derived from the technical solutions of the present invention are still within the protection scope of the present invention.

1. A device for evaluating seepage corrosion and mechanical properties of a cement sheath for cementing, comprising an acidic corrosive gas injection system, a formation water injection system, a high-pressure oil injection system, a control system and a corrosion and test system, wherein the acidic corrosive gas injection system comprises a gas cylinder, a gas booster pump and a check valve; the formation water injection system comprises a liquid storage tank, a liquid booster pump and a check valve; the gas booster pump and the liquid booster pump are connected to the control system, respectively; an outlet end of the acidic corrosive gas injection system and an outlet end of the formation water injection system are connected to the corrosion and test system, respectively; the high-pressure oil injection system comprises an oil storage tank, a high-pressure oil pump and a check valve; the high-pressure oil pump is connected to the control system; an outlet end of the high-pressure oil injection system is connected to the corrosion and test system; the corrosion and test system comprises a main body, an upper cover and a base; the main body sequentially comprises a metal cylinder, a simulated formation, a simulated casing and a center rod from outside to inside; the center rod is located in the center of the base and connected to the upper cover through a nut; the metal cylinder forms an effective seal with the simulated formation after epoxy resin is solidified; a cement sheath curing-corrosion space is formed between the simulated formation and the simulated casing; an annulus, i.e., a simulated casing-center rod annulus, is formed between the simulated casing and the center rod; a temperature control device is arranged in the annulus; the temperature control device is connected to the control system; an injection port and an oil inlet are formed in positions of the base corresponding to the simulated formation and the simulated casing-center rod annulus, respectively; the injection port is connected to the formation water injection system and the acidic corrosive gas injection system, respectively; the oil inlet is connected to the high-pressure oil injection system; a discharge port and an oil outlet are formed in positions of the upper cover corresponding to the simulated formation and the simulated casing-center rod annulus, respectively; the discharge port and the oil outlet are connected to a back pressure valve through a high-pressure pipeline, respectively; and the center rod is provided with a plurality of radial displacement meters at different positions and angles.

2. The device for evaluating seepage corrosion and mechanical properties of the cement sheath for cementing according to claim 1, wherein the acidic corrosive gas injection system and the formation water injection system provide an acidic corrosive gas and formation water for the corrosion and test system through the injection port.

3. The device for evaluating seepage corrosion and mechanical properties of the cement sheath for cementing according to claim 1, wherein the high-pressure oil injection system injects high-temperature resistant oil into the corrosion and test system through the oil inlet to control a pressure of the simulated casing-center rod annulus, thereby achieving pressure control during high-temperature and high-pressure curing and mechanical property test of the cement sheath.

4. The device for evaluating seepage corrosion and mechanical properties of the cement sheath for cementing according to claim 1, wherein the control system is used to control temperature and pressure conditions during curing and corrosion of the cement sheath for cementing, and control a pressure during the mechanical property test.

5. The device for evaluating seepage corrosion and mechanical properties of the cement sheath for cementing according to claim 1, wherein the corrosion and test system is used to cure and corrode the cement sheath under high temperature and high pressure, and evaluate mechanical properties of the cement sheath after corrosion.

6. The device for evaluating seepage corrosion and mechanical properties of the cement sheath for cementing according to claim 1, wherein the simulated formation is processed by preferably selecting outcrop rock samples according to actual formation data, or is made of artificial rock samples having parameters close to those of an actual formation.

7. A method for evaluating seepage corrosion and mechanical properties of a cement sheath for cementing by using the device according to claim 1, sequentially comprising the following steps:
(1) fixing the simulated formation in the metal cylinder by using epoxy resin, forming an effective seal after the epoxy resin is solidified, and installing the center rod, the simulated casing, and the metal cylinder containing the simulated formation on the base, respectively;
(2) injecting cement slurry into a space between the simulated formation and the simulated casing, installing the upper cover, and then tightening the upper cover by using a nut;
(3) injecting high-temperature resistant oil into the simulated casing-center rod annulus by opening the high-pressure oil injection system, injecting formation water into the simulated formation by opening the formation water injection system, and closing the back pressure valve if there is liquid flowing out of the oil outlet and the discharge port; setting a pressure of the back pressure valve, raising a temperature of the temperature control device to a required temperature through the control system, and starting to cure the cement sheath to form a simulated casing-cement sheath assembly;
(4) after the curing is completed, introducing an acidic corrosive gas H₂S/CO₂ into the corrosion and test system for corrosion testing by opening the acidic corrosive gas injection system;
(5) after the corrosion is completed, injecting a pressure into the simulated casing-center rod annulus at a certain rate through the control system and the high-pressure oil injection system to form a pressure difference between the simulated casing-cement sheath assembly and the simulated formation, recording deformation displacements generated by the simulated casing-cement sheath assembly under the pressure difference through the radial displacement meters, collecting data and drawing a stress-strain curve, and analyzing mechanical properties of the cement sheath; and
(6) reducing the temperature and pressure, unscrewing the nut, opening the upper cover and taking the cement sheath out, testing the corrosion conditions of the cement sheath and a simulated formation-cement sheath interface, and analyzing a corrosion law of the cement sheath in an H₂S/CO₂ corrosion environment.

8. The method according to claim 7, wherein the curing and the corrosion can be carried out synchronously according to the needs of experimental simulation conditions.
